SEQUENCE LISTING (10 sequences)

SEQ ID NO: 1 - protein, 721 aa, Homo sapiens
MVSVEGRAMSFQSIIHLSLDSPVHAVCVLGTEICLDLSGCAPQKCQCFTIHGSGRVLIDVANTVISEKEDATIWWPLSDPTYATVKMTSPSPSVDADKVSVTYYGPNEDAPVGTAVLYLTGIEPFGAQRSSSQSFVPLLPVSEVSQAQEAEVSLEVDIYRNGQVEMSSDKQAKKKWIWGPSGWGAILLVNCNPADVGQQLEDKKTKKVIFSEEITNLSQMTLNVQGPSCILKKYRLVLHTSKEESKKARVYWPQKDNSSTFELVLGPDQHAYTLALLGNHLKETFYVEAIAFPSAEFSGLISYSVSLVEESQDPSIPETVLYKDTVVFRVAPCVFIPCTQVPLEVYLCRELQLQGFVDTVTKLSEKSNSQVASVYEDPNRLGRWLQDEMAFCYTQAPHKTTSLILDTPQAADLDEFPMKYSLSPGIGYMIQDTEDHKVASMDSIGNLMVSPPVKVQGKEYPLGRVLIGSSFYPSAEGRAMSKTLRDFLYAQQVQAPVELYSDWLMTGHVDEFMCFIPTDDKNEGKKGFLLLLASPSACYKLFREKQKEGYGDALLFDELRADQLLSNGREAKTIDQLLADESLKKQNEYVECIHLNRDILKTELGLVEQDIIEIPQLFCLEKLTNIPSDQQPKRSFARPYFPDLLRMIVMGKNLGIPKPFGPQIKGTCCLEEKICCLLEPLGFKCTFINDFDCYLTEVGDICACANIRRVPFAFKWWKMVP

SEQ ID NO: 2 - DNA, 2166 bp, Homo sapiens
atggtcagcgtggagggccgagccatgtccttccagagtatcatccacctgtccctggacagccctgtccatgccgtttgtgtgttgggcacagaaatctgcttggatctcagcgggtgtgccccccagaagtgccagtgcttcaccatccatggctctgggagggtcttgatcgatgtggccaacacggtgatttctgagaaggaggacgccaccatctggtggcccctgtctgatcccacgtacgccacagtgaagatgacatcgcccagcccttccgtggatgcggataaggtctcggtcacatactatgggcccaacgaggatgcccccgtgggcacagctgtgctgtacctcactggcattgaaccctttggagctcagaggagctcttctcagtcctttgtcccgctgcttccagtcagtgaagtgtctcaggctcaggaggcagaggtctctctagaggtagacatctaccgcaatgggcaagttgagatgtcaagtgacaaacaggctaagaaaaaatggatctggggtcccagcggttggggtgccatcctgcttgtgaattgcaaccctgctgatgtgggccagcaacttgaggacaagaaaaccaagaaagtgatcttttcagaggaaataacgaatctgtcccagatgactctgaatgtccaaggccccagctgtatcttaaagaaatatcggctagtcctccatacctccaaggaagagtcgaagaaggcgagagtctactggccccaaaaagacaactccagtacctttgagttggtgctggggcccgaccagcacgcctataccttggccctcctcgggaaccacttgaaggagactttctacgttgaagctatagcattcccatctgccgaattctcaggcctcatctcctactctgtgtccctggtggaggagtctcaagacccgtcaattccagagactgtgctgtacaaagacacggtggtgttccgggtggctccctgtgtcttcattccctgtacccaggtgcctctggaggtttacctgtgcagggagctgcagctgcagggttttgtggacacagtgacgaagctgagtgagaagagcaacagccaggtggcatctgtctatgaggaccccaaccgcctgggcaggtggctccaggatgagatggccttctgctacacccaggctccccacaagacaacgtccttgatcctcgacacacctcaggccgccgatctcgatgagttccccatgaagtactcactgagccctggtattggctacatgatccaggacactgaggaccataaagtggccagcatggattccattgggaacctgatggtgtccccacctgtcaaggtccaagggaaagagtacccgctgggcagagtcctcattggcagcagcttttaccccagcgcagagggccgggccatgagtaagaccctccgagacttcctctatgcccagcaggtccaagcgccggtggagctctactcagattggctaatgactggccacgtggatgagttcatgtgcttcatccccacagatgacaagaatgagggcaaaaagggcttcctgctgctcctggccagccccagtgcctgctataaactgttccgagagaaacagaaggaaggctatggcgacgctcttctgtttgatgagcttagagcagatcagctcctgtctaatggaagggaagccaaaaccatcgaccaacttctggctgatgaaagcctgaagaagcagaatgaatacgtggagtgcattcacctgaaccgtgacatcctgaagacggagctgggcctggtggaacaggacatcatcgagattccccagctgttctgcttggagaagctgactaacatcccctctgaccagcagcccaagaggtcctttgcgaggccatacttccctgacctgttgcggatgattgtgatgggcaagaacctggggatccccaagccttttgggccccaaatcaaggggacctgctgcctggaagaaaagatttgctgcttgctggagcccctgggcttcaagtgcaccttcatcaatgactttgactgttacctgacagaggtcggagacatctgtgcctgtgccaacatccgccgggtgccctttgccttcaaatggtggaagatggtaccttag

SEQ ID NO: 3 - protein, 664 aa, Mus musculus
MVGMEITLDISKCAPDKCKSFTIRGSPRILIHISSSVIAGKEDTVVWRSMNHPTVALVRMVAPSPTVDEDKVLVSYFCPDQEVPTATAVLFLTGIEISLEADIYRDGQLDMPSDKQAKKKWMWGMNGWGAILLVNCSPNAVGQPDEQSFQEGPREIQNNLSQMNVTVEGPTSILQNYQLILHTSEEEAKKTRVYWSQRGSSAYELVVGPNKPVYLLPTFENRRKEAFYVEATEFPSPSFSGLISLSLSLVEKAHDECIPEIPLYKDTVMFRVAPYIFMPSTQMPLEVYLCRELQLQGFVDSVTKLSEKSKVQVVKVYEDPNRQSKWLQDEMAFCYTQAPHKTVSLILDTPRVSKLEDFPMKYTLTPGSGYLIRQIEDHRVASLDSIGNLMVSPPVKAQGKDYPLGRVLIGGSFYPSSEGRDMNKGLREFVYAQQVQAPVELFSDWLMTGHMDQFMCFVPTNDKNNDQKDFRLLLASPSACFELFEQKQKEGYGNVTLFEDIGAEQLLSNGRESKTISQILADKSFREQNTYVEKCISLNRTLLKTELGLEDKDIILIPQLFCLEQLTNVPSNQQSTKLFARPYFPDMLQIIVLGKNLGIPKPFGPKINGTCCLEEKVCGLLEPLGLKCTFIDDFDCYLANIGDVCASAIINRVPFAFKWWKMTP

SEQ ID NO: 4 - DNA, 2341 bp, Mus musculus
gggtaaggactgctgacagtggctagcttggtaagcccagccatgtcttttcagaactcactcagcctgtctctggtcaatcccacccatgccctctgcatggtaggcatggaaatcaccttggacatcagcaagtgtgcaccggacaagtgcaagtctttcaccatccgtggttcccccaggatcttgatccacatctctagctccgtcatcgctggcaaagaggacactgtggtctggaggtcaatgaaccatcccacagtggcattggtgaggatggtggcgcccagccccactgtggatgaagacaaggtgctggtctcctacttctgtcctgaccaagaagtccccacggccacagctgtgctgtttctcaccggcatcgagatctccctggaggcagacatctatcgagatggacaactggacatgccaagtgataagcaagctaagaaaaaatggatgtggggtatgaacggctggggagccatcctgcttgtgaattgtagccctaatgctgtgggccagcctgatgaacagtcctttcaggagggccccagagaaatacagaacaacctgtctcagatgaatgtaactgtggagggccccaccagcatcctacagaattaccagttgatcctacatacctccgaagaagaggcgaagaagacaagagtctactggtctcagagaggctcctctgcgtatgaactggtggtgggacccaacaagcctgtctatctcctgcctacctttgagaaccgtaggaaagaggctttctacgtagaagccacggaattcccatctcccagcttctcgggcctgatctccttgtcactctccctagtagaaaaggctcacgacgagtgcatcccagagattccgctctataaggatacagtgatgttccgggtggcaccttatatcttcatgcccagcacccagatgcctctagaggtttacctgtgcagggagctacagctgcaaggctttgtggactcagtgaccaagctgagcgagaagagcaaagtgcaggtggtaaaggtctatgaggaccccaaccgccagagcaagtggctccaggacgagatggctttctgctatactcaggctcctcacaagacggtgtcattgatccttgacaccccaagggtttccaagctggaagacttccccatgaaatacacactgacccctggctctggctacctgatccgacaaattgaggaccaccgggtggctagcctggattccatcgggaacctgatggtatctccgcctgtcaaggctcagggcaaagactaccctctagggagggtcctcattggtggcagcttttaccccagctctgagggccgggacatgaacaagggcctgcgagaattcgtgtatgcccagcaggtgcaggcccctgtggaactcttctcggactggctgatgaccggtcacatggatcaattcatgtgctttgtccctaccaatgataaaaacaacgaccagaaggacttccgcctgctgctggccagccccagtgcctgctttgagctgttcgaacagaagcagaaggaaggctatgggaacgtgaccctgtttgaagacattggagcagaacagctcctttctaatgggagggagagcaaaactatttcccaaatcctggctgacaagagttttcgagagcagaacacctatgttgagaagtgtatcagcctgaaccgcaccctcctgaagacagaactgggattggaggacaaggacatcatcctgatcccgcagctcttctgcctggagcagctgacgaatgtcccctccaaccagcagagcaccaaactcttcgcgaggccgtacttccccgacatgctgcagataatcgtgttgggcaagaaccttggaatccccaagccctttgggcccaaaatcaatggcacctgctgcctagaagagaaagtgtgtggattactggagcccctgggtctcaagtgcaccttcattgatgattttgactgctacctggccaacataggggacgtctgtgccagtgccatcataaacagggtgccatttgcattcaagtggtggaagatgaccccataaacccctggccctggcacggccagtccgcgccagtacggatggcctttgccatagatagtagtgggtgcgagcgttgttgttgcactgggtcgaagggacggaagctgggagttagggtctctcacatctaccagcttgacacttctggaggggaaaagggaaaagagcgcctatgtaaacaaattgccatagagccaataaagcatggtattctgaatacaaaaaaaaaaaaaaaaaaaaaaaaaaa

SEQ ID NO: 5 - protein, 28 aa, Homo sapiens
EPFGAQRSSSQSFVPLLPVSEVSQAQEA

SEQ ID NO: 6 - protein, 10 aa, Homo sapiens
  MOD_RES (3)..(3): METHYLATION
SGRGKGGKGC

SEQ ID NO: 7 - protein, 8 aa, Homo sapiens
  MOD_RES (4)..(4): METHYLATION
ARTKQTAR

SEQ ID NO: 8 - protein, 9 aa, Homo sapiens
  MOD_RES (5)..(5): METHYLATION
QTARKSTGV

SEQ ID NO: 9 - protein, 10 aa, Homo sapiens
  MOD_RES (1)..(1): PHOSPHORYLATION
SGRGKGGKGC

SEQ ID NO: 10 - protein, 24 aa, Mus musculus
SGGSYGGSSGGGRGGSSSGGGGVK

(19) United States
(12) Patent Application Publication
(10) Pub. No.: US 2006/0078953 A1
(43) Pub. Date: Apr. 13, 2006

(54) Epad, an oocyte specific protein

(76) Inventors: C. David Allis, New York, NY (US); John C. Herr, Charlottesville, VA (US); Scott Coonrod, New York, NY (US); Yanming Wang, New York, NY (US)

Correspondence Address: UNIVERSITY OF VIRGINIA PATENT FOUNDATION, 250 WEST MAIN STREET, SUITE 300, CHARLOTTESVILLE, VA, 22902, US

(21) Appl. No.: 10/542,178

(22) PCT Filed: Jan. 9, 2004
(86) PCT No.: PCT/US04/00591

§ 371 Date: Jul. 8, 2005

Related U.S. Application Data
(60) Provisional application No. 60/439,170, filed on Jan. 10, 2003.
(60) Provisional application No. 60/480,774, filed on Jun. 19, 2003.

(51) Int. Cl. G01N 33/537; C12N 9/16; C07K 16/40

(52) U.S. Cl. 435/7.92; 530/388.26; 435/196

(57) ABSTRACT

The present invention is directed to a human egg specific protein (ePAD), antibodies specific for the human egg specific protein and the use of the ePAD protein to identify antagonists of ePAD activity. Antagonists of ePAD activity are anticipated to have utility as female contraceptive agents.

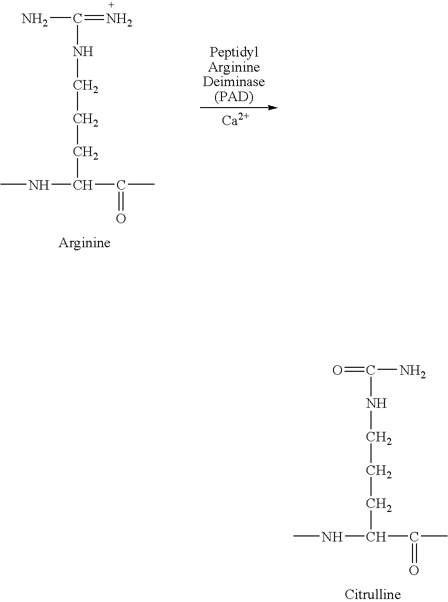

### RELATED APPLICATIONS

This application claims priority under 35 USC § 119(e) to US Provisional Application Ser. Nos. 60/439,170, filed Jan. 10, 2003, and 60/480,774, filed Jun. 19, 2003 the disclosures of which are incorporated herein by reference.

### US GOVERNMENT RIGHTS

This invention was made with United States Government support under Grant Nos. HD 38353, and U54 29099, awarded by National Institutes of Health. The United States Government has certain rights in the invention.

### BACKGROUND

There has been much interest in the development of new contraceptives for use in animals and humans. One strategy is to develop an effective contraceptive vaccine that specifically targets antigens directly involved in the fertilization process. Currently there are no vaccine formulations that are directed against egg protein(s) directly involved in the process of sperm-egg fusion step. Accordingly, one aspect of the present invention is directed to contraceptive compositions and methods that are based on egg specific proteins.

During growth, the oocyte accumulates a pool of maternal gene products and organelles required for early development. In the fully-grown egg the transcriptional machinery is silent, and after ovulation the terminally differentiated egg will die if it does not bind and fuse with a sperm. If fertilization occurs, however, maternal gene products orchestrate the transformation of the egg into a totipotent zygote within several hours. Following gamete fusion, calcium transients propagated in the egg cytoplasm lead to the activation of signal transduction cascades which are thought to mediate early embryonic epigenetic events such as remodeling of the cortical cytoskeleton, cortical granule exocytosis, completion of meiosis, polar body emission, and formation of the male and female pronuclei. Reprogramming of the parental chromatin is also thought to occur soon after fertilization and by the two-cell stage maternal transcripts begin to be replaced by embryonic transcripts, leading to the full activation of the embryonic genome. Maternal factors, however, continue to persist in the early embryo until at least the morula stage of development.

Many of the structural and molecular mechanisms mediating the physiological changes in the early embryo are as yet incompletely characterized. One of the most abundant cytoskeletal components of the mammalian egg is a fibrous network of intermediate filaments named the cytoplasmic sheets. These organelles were previously thought to be either yolk platelets or possibly ribosome storage sites, however, electron microscopic studies indicate that the highly ordered sheets are composed of parallel arrays of ˜10 nm fibers. This filamentous network is stabilized by cross-bridges and is overlain with a tightly packed particulate material. Solubility and immunological studies indicate that the Tween-20 insoluble cross-linked fibers contain keratin (but not vimentin or tubulin) and the soluble fraction largely consists of an unidentified ˜69 kDa protein. Soluble protein kinase C associates with the cytoplasmic sheets, phosphorylates cytokeratin and the ˜69 kDa soluble protein, and may be responsible for initiating the changes in spatial organization that these sheets undergo at the time of fertilization. Cytoplasmic sheets arise during oocyte development, are unique to the egg and early embryo, are conserved amongst mammals, and undergo extensive spatial re-organizations during the critical developmental transitions of fertilization, compaction and blastulation.

Peptidylarginine deiminases (PADs) represent a family of calcium-dependent sulfhydryl enzymes that convert arginine residues to citruline in proteins. PAD activity appears to be upregulated by a variety of estrogenic compounds and to date, four types of PADs have been characterized, with each differing in its pattern of substrate specificity and tissue distribution. For example, the widely distributed and well characterized type II PAD is especially abundant in muscle and brain and is associated with deimination of myelin basic protein. PAD V, found in granulocyte-differentiated HL-60 cells, is thought to play a role in myeloid cell differentiation, and likely targets nucleophosmin and histone core proteins for deimination. Type I and m PADs have been characterized in the epidermis; with type m PAD being found to deiminate trichohyalin in hair follicles and Type I PAD deiminating keratin and filaggrin during epidermal differentiation. It has been suggested that the deimination of keratin and filaggrin in the epidermis induces changes in the spatial organization of keratin intermediate filaments during keratinocyte maturation. Deiminated keratin has been identified in day 18 embryos, however, the presence of deiminated keratin at earlier stages of development has not been investigated.

An oocyte specific, peptidylarginine deiminase-like protein has been previously described in International Application No. PCT/US01/01718 filed Jan. 19, 2001, the disclosure of which is incorporated herein. Due to the protein's 40% identity with the peptidylarginine deiminase enzyme family, the protein has been named ePAD, for egg and embryo abundant PAD. This protein is expressed in primary oocytes and persists until at least the blastocyst stage of development.

At the ultrastructural level, ePAD has been found to localize to the egg's cytoplasmic sheets. Thus applicants propose that ePAD associated arginine deiminase reactions directed against cytokeratin and possibly other proteins, results in reorganization of the cytoskeleton during early development. According to this proposed function of ePAD, this protein makes an attractive target for isolating contraceptive agents that interfere with its activity.

In addition, posttranslational modifications of histone amino-termini have long been thought to play a central role in the control of chromatin structure and function. In particular, evidence is emerging that histone proteins, and their associated covalent modifications, contribute to a mechanism that can alter chromatin structure, thereby leading to inherited differences in transcriptional “on-off” states or to the stable propagation of epigenetic information by defining a specialized higher-order structure (see International Application No. PCT/US01/26283, the disclosure of which is incorporated herein). Reprogramming of the parental chromatin via histone modifications is thought to occur soon after fertilization. One main prediction is that the histone “marks” are erased prior to embryogenesis in order to reinstate the totipotency of the embryo. The mechanism for resetting the histone code is unknown, however, as described herein it is anticipated that ePAD plays a role in such a mechanism.

### SUMMARY OF VARIOUS EMBODIMENTS OF THE INVENTION

The present invention is directed to a human egg specific protein (ePAD), antibodies specific for the human egg specific protein and nucleic acid sequences encoding said protein, as well as compositions comprising such compounds. In one embodiment the ePAD protein and nucleic acid sequences are used as components in a contraceptive vaccine. Compositions comprising the amino acid, nucleic acid or antibodies of the present invention can also be used in accordance with the present invention as diagnostic indicators of fertility.

### BRIEF DESCRIPTION OF THE DRAWINGS

FIG. 1 is a schematic representation of PAD enzymatic conversion of protein-contained arginine to citruline.

FIG. 2. provides an alignment of the amino acid sequences of human ePAD (SEQ ID NO: 1) and mouse ePAD (SEQ ID NO: 3). As seen in FIG. 2 the human ePAD amino acid sequence contains a 28 amino acid sequence (SEQ ID NO: 5) located near the N-terminus that is absent in the mouse ePAD sequence.

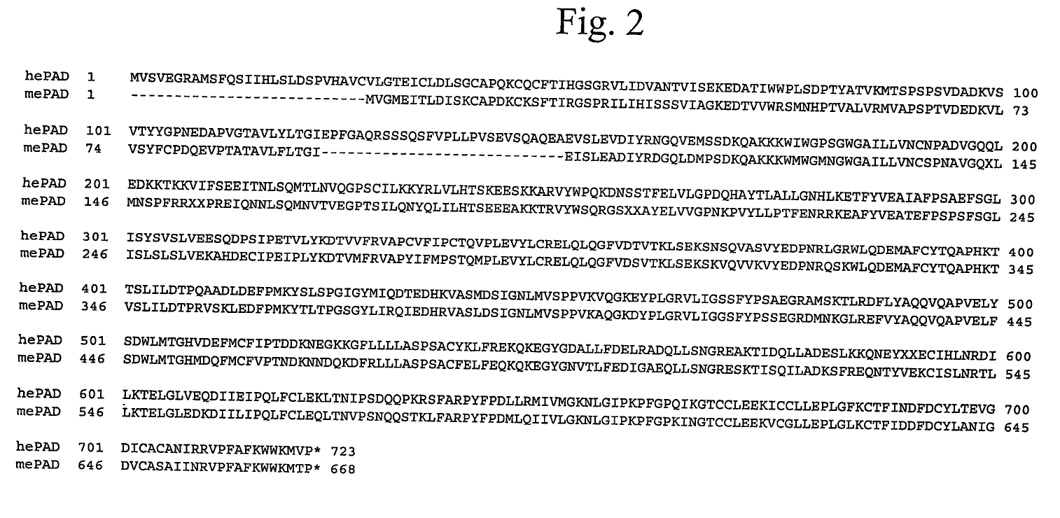

### DETAILED DESCRIPTION OF EMBODIMENTS

Definitions

In describing and claiming the invention, the following terminology will be used in accordance with the definitions set forth below.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

A polylinker is a nucleic acid sequence that comprises a series of three or more closely spaced restriction endonuclease recognitions sequences.

“Operably linked” refers to a juxtaposition wherein the components are configured so as to perform their usual function. Thus, control sequences or promoters operably linked to a coding sequence are capable of effecting the expression of the coding sequence.

As used herein, “nucleic acid,” “DNA,” and similar terms also include nucleic acid analogs, i.e. analogs having other than a phosphodiester backbone. For example, the so-called “peptide nucleic acids,” which are known in the art and have peptide bonds instead of phosphodiester bonds in the backbone, are considered within the scope of the present invention.

The term “peptide” encompasses a sequence of 3 or more amino acids wherein the amino acids are naturally occurring or synthetic (non-naturally occurring) amino acids. Peptide mimetics include peptides having one or more of the following modifications:

1. peptides wherein one or more of the peptidyl —C(O)NR— linkages (bonds) have been replaced by a non-peptidyl linkage such as a —CH₂-carbamate linkage (—CH₂OC(O)NR—), a phosphonate linkage, a —CH₂-sulfonamide (—CH₂—S(O)₂ NR—) linkage, a urea (—NHC(O)NH—) linkage, a—CH₂-secondary amine linkage, or with an alkylated peptidyl linkage (—C(O)NR—) wherein R is C₁-C₄ alkyl;

2. peptides wherein the N-terminus is derivatized to a —NRR₁ group, to a —NRC(O)R group, to a —NRC(O)OR group, to a —NRS(O)₂R group, to a —NHC(O)NHR group where R and R₁ are hydrogen or C₁-C₄ alkyl with the proviso that R and R₁ are not both hydrogen;

3. peptides wherein the C terminus is derivatized to —C(O)R₂ where R₂ is selected from the group consisting of C₁-C₄ alkoxy, and —NR₃R₄ where R₃ and R₄ are independently selected from the group consisting of hydrogen and C₁-C₄ alkyl.

Naturally occurring amino acid residues in peptides are abbreviated as recommended by the IUPAC-IUB Biochemical Nomenclature Commission as follows: Phenylalanine is Phe or F; Leucine is Leu or L; Isoleucine is Ile or I; Methionine is Met or M; Norleucine is Nle; Valine is Val or V; Serine is Ser or S; Proline is Pro or P; Threonine is Thr or T; Alanine is Ala or A; Tyrosine is Tyr or Y; Histidine is His or H; Glutamine is Gin or Q; Asparagine is Asn or N; Lysine is Lys or K; Aspartic Acid is Asp or D; Glutamic Acid is Glu or E; Cysteine is Cys or C; Tryptophan is Trp or W; Arginine is Arg or R; Glycine is Gly or G, and X is any amino acid. Other naturally occurring amino acids include, by way of example, 4-hydroxyproline, 5-hydroxylysine, and the like.

Synthetic or non-naturally occurring amino acids refer to amino acids which do not naturally occur in vivo but which, nevertheless, can be incorporated into the peptide structures described herein. The resulting “synthetic peptide” contains amino acids other than the 20 naturally occurring, genetically encoded amino acids at one, two, or more positions of the peptides. For instance, naphthylalanine can be substituted for trytophan to facilitate synthesis. Other synthetic amino acids that can be substituted into peptides include L-hydroxypropyl, L-3,4-dihydroxyphenylalanyl, alpha-amino acids such as L-alpha-hydroxylysyl and D-alpha-methylalanyl, L-alpha.-methylalanyl, beta.-amino acids, and isoquinolyl. D amino acids and non-naturally occurring synthetic amino acids can also be incorporated into the peptides. Other derivatives include replacement of the naturally occurring side chains of the 20 genetically encoded amino acids (or any L or D amino acid) with other side chains.

As used herein, the term “conservative amino acid substitution” is defined herein as an amino acid exchange within one of the following five groups:
I. Small aliphatic, nonpolar or slightly polar residues:
   Ala, Ser, Thr, Pro, Gly;
II. Polar, negatively charged residues and their amides:
   Asp, Asn, Glu, Gln;
III. Polar, positively charged residues:
   His, Arg, Lys;
IV. Large, aliphatic, nonpolar residues:
   Met Leu, Ile, Val, Cys
V. Large, aromatic residues:
   Phe, Tyr, Trp

As used herein, the term “antibody” refers to a polyclonal or monoclonal antibody or a binding fragment thereof such as Fab, F(ab′)2 and Fv fragments.

As used herein, the term “ePAD antibody” refers to an antibody that specifically binds to the amino acid sequence of SEQ ID NO: 1 or SEQ ID NO: 3.

As used herein, the term “biologically active fragments” or “bioactive fragment” of an ePAD polypeptide encompasses natural or synthetic portions of the full-length protein that are capable of specific binding to their natural ligand.

The term “non-native promoter” as used herein refers to any promoter that has been operably linked to a coding sequence wherein the coding sequence and the promoter are not naturally associated (i.e. a recombinant promoter/coding sequence construct).

As used herein, a transgenic cell is any cell that comprises a nucleic acid sequence that has been introduced into the cell in a manner that allows expression of a gene encoded by the introduced nucleic acid sequence.

As used herein, the term “treating” includes alleviating the symptoms associated with a specific disorder or condition and/or preventing or eliminating said symptoms. For example, treating cancer includes preventing or slowing the growth and/or division of cancer cells as well as killing cancer cells.

### Embodiments

As described in International Application No. PCT/US01/01718 (the disclosure of which is incorportated herein), ePAD is one of the most abundant proteins yet to be characterized in the mouse egg. This highly abundant egg and embryo protein is expressed from the primary oocyte stage of oogenesis until at least the blastocyst stage of development. At the ultrastructural level, ePAD localizes to the egg cytoplasmic sheets, keratin containing structures known to undergo changes in their structure during early development.

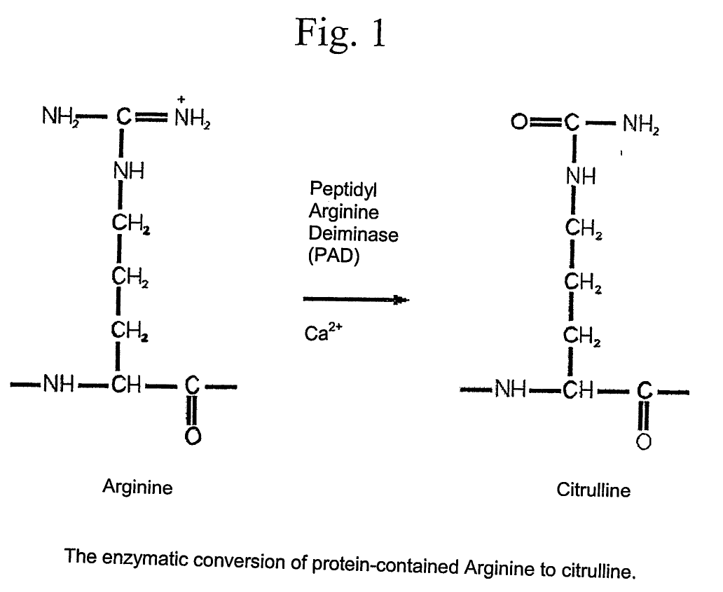

The nucleic acid sequences of human and mouse ePAD are shown as SEQ ID NO: 2 and SEQ ID NO: 4, respectively and the deduced human and mouse amino acid sequences are shown as SEQ ID NO: 1 and SEQ ID NO: 3, respectively. Blast homology search demonstrates that mouse ePAD is most similar (40% identical, 60% positive, 5% gaps) to the peptidylarginine deiminase (PAD) family of enzymes. PADs are post-translation modification enzymes which convert arginine residues on proteins to citruline residues in the presence of calcium (see FIG. 1). The human and mouse ePAD genes are both present as single copy genes in their respective genome and the mouse and human proteins share approximately 61% sequence identity and 76% conserved sequence identity.

In accordance with one embodiment of the present invention a purified polypeptide is provided comprising the amino acid sequence of SEQ ID NO: 1, or an amino acid sequence that differs from SEQ ID NO: 1 by one or more conservative amino acid substitutions. The polypeptides of the present invention may include additional amino acid sequences to assist in the stabilization and/or purification of recombinantly produced polypeptides. These additional sequences may include intra- or inter-cellular targeting peptides or various peptide tags known to those skilled in the art. In one embodiment, the purified polypeptide comprises an amino acid of SEQ ID NO: 1 and a peptide tag, wherein the peptide tag is linked to the ePAD peptide sequence. Suitable expression vectors for expressing such fusion proteins and suitable peptide tags are known to those skilled in the art and commercially available. In one embodiment the tag comprises a His tag.

The present invention also encompasses nucleic acid sequences that encode human ePAD. In one embodiment a purified nucleic acid sequence is provided comprising the sequence of SEQ ID NO: 2 or a fragment thereof. The present invention also encompasses recombinant human ePAD gene constructs. In one embodiment, the recombinant gene construct comprises a non-native promoter operably linked to a nucleic acid sequence comprising SEQ ID NO: 2. The non-native promoter is preferably a strong constitutive promoter that allows for expression in a predetermined host cell. These recombinant gene constructs can be introduced into host cells to produce transgenic cell lines that synthesize the ePAD gene products. Host cells can be selected from a wide variety of eukaryotic and prokaryotic organisms, and two preferred host cells are E. coli and yeast cells.

In accordance with one embodiment, a nucleic acid sequence comprising SEQ ID NO: 2 is inserted into a eukaryotic or prokaryotic expression vector in a manner that operably links the gene sequence to the appropriate regulatory sequences, and human ePAD is expressed in the eukaryotic or prokaryotic host cell. In one embodiment the gene construct comprises the nucleic acid sequence of SEQ ID NO: 2 or SEQ ID NO: 4 operably linked to a eukaryotic promoter. Suitable eukaryotic host cells and vectors are known to those skilled in the art. The baculovirus system is also suitable for producing transgenic cells and synthesizing the ePAD genes of the present invention. One aspect of the present invention is directed to transgenic cell lines that contain recombinant genes that express human ePAD and fragments of the human ePAD coding sequence. As used herein a transgenic cell is any cell that comprises an exogenously introduced nucleic acid sequence.

In one embodiment the introduced nucleic acid is sufficiently stable in the transgenic cell (i.e. incorporated into the cell's genome, or present in a high copy plasmid) to be passed on to progeny cells. The cells can be propagated in vitro using standard cell culture procedure, or in an alternative embodiment, the host cells are eukaryotic cells and are propagated as part of a non-human animal, including for example, a non-human transgenic animal. In one embodiment the transgenic cell is a human cell propagated in vitro and comprises the nucleic acid sequence of SEQ ID NO:2.

The present invention also encompasses a method for producing human and mouse ePAD. The method comprises the steps of introducing a nucleic acid sequence comprising a sequence that encodes the human or mouse ePAD into a host cell, and culturing the host cell under conditions that allow for expression of the introduced human ePAD gene. In one embodiment the promoter is a conditional or inducible promoter, alternatively the promoter may be a tissue specific or temporal restricted promoter (i.e. operably linked genes are only expressed in a specific tissue or at a specific time). The synthesized ePAD can be purified using standard techniques and used in high throughput sceeens to identify inhibitors of ePAD activity. Alternatively, in one embodiment the recombinantly produced ePAD polypeptides, or fragments thereof are used to generate antibodies against the human or mouse ePAD. The recombinanatly produced ePAD proteins can also be used to obtain crystal structures. Such structures would allow for crystallography analysis that would lead to the design of specific drugs to inhibit ePAD function.

The entire mouse cDNA sequence was used to probe a Northern blot containing poly-(A)+ mRNA isolated from COC, ovary, heart, brain, spleen, lung, liver, small intestine, kidney and testis. The cumulus oocyte complex (COC) lane represented mRNA isolated from ovulated eggs and included support cells and tissues affiliated with recently ovulated eggs. The Northern blot analysis demonstrated that mouse ePAD mRNA is abundantly expressed in the ovary, and at longer exposure of the Northern blot ePAD mRNA expression was also detected in the testes albeit at a much lesser extent.

Immunofluorescent localization of ePAD in human eggs and an 8 cell human embryos was conducted using antibodies to mouse recombinant ePAD (see Example 1 and 2). Cytoplasmic staining was observed in metaphase II eggs and in 8-cell embryos incubated with ePAD IgG and no staining was evident when eggs/embryos were incubated with preimmune IgG. Furthermore, cytoplasmic staining of primary follicles can be seen in ovary cross-sections incubated with ePAD sera. Again, no staining was visible when ovary cross-sections were incubated with the pre-immune sera. Thus indirect immunofluorescence reveals that ePAD is abundant in the cytoplasm of human oocytes and embryos, and is present in primary follicles in human ovarian tissue.

The developmental expression pattern of human ePAD and its association with the egg cytoplasmic sheets, suggest that this protein plays a role in cellular cytostructure and thus serves as a target for identifying contraceptive agents. Previous experiments have shown that the cytoplasmic sheets consist of a highly cross-linked network of Triton X-100 detergent insoluble intermediate filaments that are coated with a Tween-20 detergent soluble protein component. The most abundant soluble component of the sheets is a ˜69 kDa protein that has not been characterized at the molecular level. Given ePADs localization to the cytoskeletal sheets, its solubility characteristics, and its molecular weight (75 vs. 69 kDa), it is tempting to speculate that ePAD is the soluble ˜69 kDa protein previously described in the literature. The molecular nature of the insoluble component has been partially characterized and shown to contain cytokeratin. Because keratin is such a well characterized PAD substrate in epithelial cells, it is anticipated to be a substrate for ePAD in eggs and embryos.

In epithelial cells, deimination of specific arginine residues in keratin and filaggrin is known to be involved in the conversion of a pre-existing fine cytokeratin network to more densely bundled linear filamentous structures. This reorganization is reminiscent of changes that have been described in the cytoskeletal sheets in the egg where the preexisting multi-lamellar, whorl-like pattern in unfertilized eggs, becomes more linearly arranged in the fertilized zygote. At compaction, the sheets appear as highly polarized structures with one end anchored on the apical plasma membrane and extending downward toward the basolateral surface within each blastomere. As with ePAD protein expression, by the blastocyst stage of development the sheet density begins to diminish. In the trophectoderm, the sheets are localized near the plasma lamina while in the inner cell mass blastomeres the sheets tend to be more centrally located.

Because PADs are calcium-dependent enzymes, fertilization represents a particularly interesting developmental stage for potential ePAD activity since large calcium transients are known to occur following sperm-egg fusion. Following fertilization, one could imagine that the calcium transients could activate ePAD, which would then deiminate keratin resulting in the observed cytoskeletal sheet reorganization. Such reorganization may be essential for sperm internalization or polar body extrusion.

In addition to the keratins of the cytoskeletal sheets, other proteins also represent potential ePAD deimination targets at fertilization. For example, protamine, an arginine-rich (˜60% arginine residues) sperm-specific histone-like protein, is known to be an excellent in vitro substrate for PADs (Sugawara et al., 1982, J. Biochem (Tokyo) 99, 1417-24). Given that substrate deimination causes changes in secondary structure, deimination of sperm protamine by ePAD, following gamete fusion might facilitate nuclear decondensation. In fact, as reported herein, commercially available skeletal muscle PADs readily decondense sperm chromatin in vitro. Furthermore, since histones are a substrate for PAD, ePAD is also anticipated to interact specifically with gamete histones, and thus may be involved with decondensing the sperm nucleus upon fusion of the egg and sperm.

Accordingly, one aspect of the present invention is directed to the isolation of agents that inhibit ePAD activity and thus serve as contraceptive agents. In accordance with one embodiment, human and mouse ePAD gene products are used to screen for specific inhibitors of ePAD enzymatic activity (such as inhibition of ePADs deiminase activity). These inhibitors will be used in accordance with the present invention either alone or in conjunction with other contraceptive agents to prevent unintended pregnancies.

In accordance with one embodiment a method for identifying compounds that inhibit the enzymatic activity of ePAD is provided. In one embodiment the method comprises the steps of contacting human ePAD protein with a methylated peptide substrate in the presence of a potential ePAD inhibitor for a predetermined length of time, measuring the amount of demethylated peptide produced (relative to the remaining methylated peptide) and comparing that amount with the amount of demethylated peptide produced when the substrate is contacted with human ePAD protein in the absence of the potential ePAD inhibitor. The length of time for contacting the substrate with the ePAD protein is dependant on the amount of ePAD protein present in the sample relative to the concentration of the substrate. Typically the predetermined length of time is selected such that in the absence of inhibitors and under optimal conditions (temperature, pH, etc.) more that 75% of the substrate would be demethylated after the set amount of time. The relative amount of demethylated peptide can be determined either through the use of labeled methylated substrates (wherein demethlation removes the label) or through the use of antibodies that are specific for either the methylated or non-methylated peptides. In one embodiment the methylated peptide substrate comprises a peptide selected from the group consisting of SGR*GKGGKGC (SEQ ID NO: 6), ARTK*QTAR (SEQ ID NO: 7) and QTARK*STGV (SEQ ID NO: 8), wherein the * represents a methylated residue. Antibodies that are specific for the methylated peptides have been previously described in PCT/US01/26283, the disclosure of which is encorporated herein.

In another embodiment inhibitors of ePAD activity can be identified by measuring the conversion of peptiylarginine to citruline. In accordance with this embodiment the method comprises the steps of contacting human ePAD protein with a peptiylarginine peptide substrate in the presence of a potential ePAD inhibitor for a predetermined length of time, and measuring the amount of citruline peptide produced. The amount of citruline produced in the presence of the potential inhibitor is then compared to the amount of citruline peptide produced when the substrate is contacted with human ePAD protein in the absence of the potential ePAD inhibitor.

In accordance with another embodiment of the present invention an antigenic composition is provided comprising a purified the amino acid sequence of SEQ ID NO: 1, or an antigenic fragment thereof. The composition can be combined with a pharmaceutically acceptable carrier or adjuvant and administered to a mammalian species to induce an immune response. Such compositions have utility for raising antibodies agains the ePAD protein, and in one embodiment are used as contraceptive vaccine formulations. The vaccines of the invention may be multivalent or univalent. Multivalent vaccines are made from recombinant viruses/vectors that direct the expression of more than one antigen.

In one aspect of the invention, a polypeptide comprising the amino acid sequence of SEQ ID NO: 1, or an antigenic fragment thereof is delivered to a subject to elicit an active immune response. The immune response induced by the antigenic composition acts as a temporary and reversible antagonist of the function of the ePAD protein. For example, such antigenic compositions could be used for active immunization of a subject, to raise an antibody response to temporarily block the sperm's access to the egg plasma antigen. In one aspect of the invention, an antigen could be administered at a certain period of the month, for example during ovulation of a female subject to block fertilization.

In another aspect of the invention, a polypeptide comprising the amino acid sequence of SEQ ID NO: 1, or an antigenic fragment thereof is used as a vaccine for permanent sterilization of a subject. Such vaccines can be used to elicit a T-cell mediated attack on the eggs, having an othoritic effect, useful as a method for irreversible sterilization. Methods for generating T-cell specific responses, such as adoptive immunotherapy, are well known in the art (see, for example, Vaccine Design, Michael F. Powell and Mark J. Newman Eds., Plenum Press, New York, 1995, pp 847-867). Such techniques may be particular useful for vetinary contraceptive or sterilization purposes, where a single dose vaccination may be desirable.

Suitable preparations of vaccines include injectables, either as liquid solutions or suspensions; solid forms suitable for solution (or suspension) in liquid prior to injection, may also be prepared. The preparation may also be emulsified, or the polypeptides encapsulated in liposomes. The active immunogenic ingredients are often mixed with excipients which are pharmaceutically acceptable and compatible with the active ingredient. Suitable excipients are, for example, water saline, dextrose, glycerol, ethanol, or the like and combinations thereof. In addition, if desired, the vaccine preparation may also include minor amounts of auxiliary substances such as wetting or emulsifying agents, pH buffering agents, and/or adjuvants which enhance the effectiveness of the vaccine.

Examples of adjuvants which may be effective, include, but are not limited to: mineral gels, e.g., aluminum hydroxide, surface active substances such as lysolecithin, pluronic polyols; polyanions; peptides; oil emulsions; alum, and MDP; N-acetyl-muramyl-L-threonyl-D-isoglutamine (thr-MDP), N-acetyl-nor-muramyl-L-alanyl-D-isoglutamine, N-acetylmuramyl-L-alanyl-D-isoglutaminyl-L-alanine-2-(1′-2′-dipalmitoyl-sn-glycero-3-hydroxyphosphoryloxy)-ethylamine, aluminum hydroxide;

The effectiveness of an adjuvant may be determined by measuring the induction of antibodies directed against an immunogenic polypeptide comprising a sequence from the protein of SEQ ID NO: 1, relative to the antibodies resulting from administration of this polypeptide in compositions comprising the various adjuvants. Effective doses (immunizing amounts) of the vaccines of the invention may also be extrapolated from dose-response curves derived from animal model test systems.

The polypeptides may be formulated into the vaccine as neutral or salt forms. Pharmaceutically acceptable salts include the acid addition salts (formed with free amino groups of the peptide) and which are formed with inorganic acids, such as, for example, hydrochloric or phosphoric acids, or organic acids such as acetic, oxalic, tartaric, maleic, and the like. Salts formed with free carboxyl groups may also be derived from inorganic bases, such as, for example, sodium potassium, ammonium, calcium, or ferric hydroxides, and such organic bases as isopropylamine, trimethylamine, 2-ethylamino ethanol, histidine, procaine and the like.

The present invention provides a method of immunizing an animal, comprising administering to the animal an effective immunizing dose of an ePAD containing antigenic composition of the present invention. In one embodiment the antigenic composition comprises an amino acid sequence of SEQ ID NO: 1 by itself, or in combination with other egg specific antigens. The immunogen may also be incorporated into liposomes, or conjugated to polysaccharides and/or other polymers for use in a vaccine formulation. In instances where the recombinant antigen is a hapten, i.e., a molecule that is antigenic in that it can react selectively with cognate antibodies, but not immunogenic in that it cannot elicit an immune response, the hapten may be covalently bound to a carrier or immunogenic molecule; for instance, a large protein such as serum albumin will confer immunogenicity to the hapten coupled to it. The patient to which the vaccine is administered is preferably a mammal, most preferably a human, but can also be a non-human animal including but not limited to cows, horses, sheep, pigs, fowl (e.g., chickens), goats, cats, dogs, hamsters, mice and rats.

The present invention also encompasses antagonists and agonists, including compounds or nucleotide constructs that inhibit expression or the activity of human ePAD (i.e. transcription factor inhibitors, antisense, interfering oligonucleotides and ribozyme molecules, or gene or regulatory sequence replacement constructs) as well as antibodies that interfere with the activity of ePAD. The human ePAD (HePAD) gene includes nucleic acids that comprise the sequence of SEQ ID NO: 2 as well as other human gene family members or derivative thereof.

In accordance with one embodiment of the present invention an antibody is provided that specifically binds to the human and/or mouse ePAD polypeptide. In accordance with one embodiment an antibody is provided that specifically binds to the polypeptide of SEQ ID NO: 1. Antibodies generated in accordance with the present invention may include, but are not limited to, polyclonal, monoclonal, chimeric (i.e “humanized” antibodies), single chain (recombinant), Fab fragments, and fragments produced by a Fab expression library. These antibodies can be used as diagnostic agents for the diagnosis of conditions or diseases characterized by in appropriate expression or overexpression of ePAD, or in assays to monitor the effectiveness of an ePAD agonist, antagonist or inhibitor. The antibodies may be used with or without modification, and may be labeled by joining them, either covalently or non-covalently, with a reporter molecule. In addition, the antibodies can be formulated with standard carriers and optionally labeled to prepare therapeutic or diagnostic compositions.

In accordance with one embodiment, antibodies are provided that bind to human ePAD without binding to other human epitopes (including other human PADs), and in one embodiment an antibody is provided that specifically binds to the amino acid sequence of SEQ ID NO: 1 without binding to the mouse ePAD sequence of SEQ ID NO: 3. In particular, the amino acid sequence of SEQ ID NO: 1, or analog or fragment thereof, may be used as an immunogen to generate antibodies which immunospecifically bind such an immunogen. In one embodiment of the present invention an antigenic compound is provided for generating antibodies, wherein the compound comprises the amino acid sequence of EPFGAQRSSSQSFVPLLPVSEVSQAQEA (SEQ ID NO: 5) or a fragment thereof. Antibodies raised against human ePAD can be generated using standard techniques, and include, but are not limited to, polyclonal, monoclonal, chimeric, single chain, Fab fragments, and Fab expression libraries. The antibodies generated can be formulated with standard carriers and optionally labeled to prepare therapeutic or diagnostic compositions. In one embodiment, a composition is provided comprising an ePAD specific antibody and a pharmaceutically acceptable carrier. In one embodiment the composition further comprises a surfactant, adjuvant, excipient or stabilizer. In general, water, saline, aqueous dextrose, and related sugar solution, and glycols such as, propylene glycol or polyethylene glycol, are preferred liquid carriers, particularly for injectable solutions.

Various procedures known in the art may be used for the production of polyclonal antibodies to human ePAD or derivatives or analogs thereof. For the production of antibody, various host animals, including but not limited to rabbits, mice, rats, etc can be immunized by injection with the polypeptide of SEQ ID NO: 1, or a synthetic version, or derivative (e.g., fragment) thereof. Various adjuvants may be used to increase the immunological response, depending on the host species, and including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum.

For preparation of monoclonal antibodies directed toward the sequence of SEQ ID NO: 1, or fragment thereof, any technique which provides for the production of antibody molecules by continuous cell lines in culture may be used. For example, the hybridoma technique originally developed by Kohler and Milstein (1975, Nature 256:495-497), as well as the trioma technique, the human B-cell hybridoma technique (Kozbor et al., 1983, Immunology Today 4:72), and the EBV-hybridoma technique to produce human monoclonal antibodies (Cole et al., 1985, in Monoclonal Antibodies and Cancer Therapy, Alan R Liss, Inc., pp. 77-96). In an additional embodiment of the invention, monoclonal antibodies can be produced in germ-free animals utilizing recent technology (PCT/US90/02545). According to the invention, human antibodies may be used and can be obtained by using human hybridomas (Cote et al., 1983, Proc. Natl. Acad. Sci. U.S.A. 80:2026-2030) or by transforming human B cells with EBV virus in vitro (Cole et al., 1985, in Monoclonal Antibodies and Cancer Therapy, Alan R. Liss, pp. 77-96). In one embodiment, techniques developed for the production of “chimeric antibodies” (Morrison et al., 1984, Proc. Natl. Acad. Sci. U.S.A. 81:6851-6855; Neuberger et al., 1984, Nature 312:604-608; Takeda et al., 1985, Nature 314:452-454) by splicing the genes from a mouse antibody molecule specific for egg surface proteins together with genes from a human antibody molecule of appropriate biological activity can be used; such “humanized” antibodies are within the scope of this invention.

According to the present invention, techniques described for the production of single chain antibodies (U.S. Pat. No. 4,946,778) can be adapted to produce human ePAD single chain antibodies. An additional embodiment of the invention utilizes the techniques described for the construction of Fab expression libraries (Huse et al., 1989, Science 246:1275-1281) to allow rapid and easy identification of monoclonal Fab fragments with the desired specificity for human ePAD, derivatives, or analogs.

Antibody fragments which contain the idiotype of the molecule can be generated by known techniques. For example, such fragments include but are not limited to: the F(ab′)₂ fragment which can be produced by pepsin digestion of the antibody molecule; the Fab′ fragments which can be generated by reducing the disulfide bridges of the F(ab′)₂ fragment, the Fab fragments which can be generated by treating the antibody molecule with papain and a reducing agent, and Fv fragments.

In the production of antibodies, screening for the desired antibody can be accomplished by techniques known in the art, e.g. ELISA (enzyme-linked immunosorbent assay). For example, to select antibodies which recognize a specific domain of human ePAD, one may assay generated hybridomas for a product which binds to a peptide containing such domain. For selection of an antibody that specifically binds human ePAD but which does not specifically bind to mouse ePAD, one can select on the basis of positive binding to human ePAD and a lack of binding to mouse ePAD. Furthermore, peptide antigenic fragments unique to the human ePAD sequence can be selected to further the likelihood of generating human specific ePAD antibodies.

The foregoing antibodies can be used in methods known in the art relating to the localization and activity of human ePAD, e.g., for imaging these proteins, measuring levels thereof in appropriate physiological samples, in diagnostic methods, etc. The antibodies generated against ePAD antigens can also be used as contraceptive or sterilization agents (i.e. passive immunotherapy), or for use in diagnostic immunoassays or the generation of antiidiotypic antibodies. For example, in one embodiment ePAD antibodies are isolated (e.g., immunoaffinity chromatography, centrifugation, precipitation, etc.) and used in diagnostic immunoassays, or the antibodies may be used to monitor treatment and/or disease progression. Any immunoassay system known in the art, such as those listed supra, may be used for this purpose including but not limited to competitive and noncompetitive assay systems using techniques such as radioimmunoassays, ELISA (enzyme-linked immunosorbent assays), “sandwich” immunoassays, precipitin reactions, gel diffusion precipitin reactions, immunodiffusion assays, agglutination assays, complement-fixation assays, immunoradiometric assays, fluorescent immunoassays, protein A immunoassays and immunoelectrophoresis assays.

In one embodiment the antibodies generated against ePAD are used in passive immunotherapy as a means of contraception. In one embodiment the antibodies are specific for an amino acid sequence comprising SEQ ID NO: 1, or a fragment thereof, including for example SEQ ID NO: 5. In one embodiment the antibodies are humanized using standard techniques known to those skilled in the art. Advantageously, this method provides a short-term contraceptive effect resulting from the administration of pre-formed antibodies directed against ePAD. The ePAD antibodies can also be used in the production of antiidiotypic antibody. The antiidiotypic antibody can then in turn be used for immunization, in order to produce a subpopulation of antibodies that bind natural ligands of ePAD (Jerne, 1974, Ann. Immunol. (Paris) 125c:373; Jerne, et al., 1982, EMBO J. 1:234).

The vaccine and passive immunity formulations of the invention comprise an effective immunizing amount of an ePAD antigen or anti-ePAD antibodies, respectively, and a pharmaceutically acceptable carrier or excipient. In one embodiment the antigen is a peptide comprising the amino acid sequence of SEQ ID NO: 1. Pharmaceutically acceptable carriers are well known in the art and include but are not limited to saline, buffered saline, dextrose, water, glycerol, sterile isotonic aqueous buffer, and combinations thereof. One example of such an acceptable carrier is a physiologically balanced culture medium containing one or more stabilizing agents such as stabilized, hydrolyzed proteins, lactose, etc. The carrier is preferably sterile. The formulation should suit the mode of administration.

The composition, if desired, can also contain minor amounts of wetting or emulsifying agents, or pH buffering agents. The composition can be a liquid solution, suspension, emulsion, tablet, pill, capsule, sustained release formulation, or powder. Oral formulation can include standard carriers such as pharmaceutical grades of mannitol, lactose, starch, magnesium stearate, sodium saccharine, cellulose, magnesium carbonate, etc.

Generally, the ingredients are supplied either separately or mixed together in unit dosage form, for example, as a dry lyophilized powder or water free concentrate in a hermetically sealed container such as an ampoule or sachette indicating the quantity of active agent. Where the composition is administered by injection, an ampoule of sterile diluent can be provided so that the ingredients may be mixed prior to administration.

The precise dose of the contraceptive composition to be employed in the formulation will also depend on the route of administration, and the nature of the patient, and should be decided according to the judgment of the practitioner and each patient's circumstances according to standard clinical techniques. An effective immunizing amount is that amount sufficient to produce an immune response to the antigen in the host to which the vaccine preparation is administered.

Development of a contraceptive vaccine based on egg antigens will require the means to quickly and easily assess the effectiveness of the vaccine. An assay that can specifically measure immune responses to egg molecules will be useful to assess, although indirectly, the fertility status of vaccinated individuals. Such a test for assaying egg specific antibody titre levels in an individual would be effective for both single- or multiple-antigen vaccines. In one embodiment, recombinant ePAD molecules are used to develope a diagnostic test for specific forms of infertility based on the detection of autoantibodies.

Another embodiment of the present invention is directed to small molecule inhibitors of ePAD and their use to decrease the fertility of a female mammal. In one embodiment a method of female contraception is provided wherein said method comprises the steps of inhibiting the activity of human ePAD. In one embodiment the fertility of a female mammal is decreased by the administration of a pharmaceutical composition that comprises an agent that specifically interferes with ePAD activity. In one embodiment the fertility inhibiting composition comprises a chemical entity that specifically inhibits the demethylase activity of ePAD. In another embodiment the inhibitory composition comprises an antibody specific for ePAD, and in one embodiment the antibody specifically binds to the amino acid sequence of SEQ ID NO: 1. Alternatively, the composition may comprises an antisense or interference RNA that prevents or disrupts the expression or activity of ePAD in an animal. In accordance with one embodiment the fertility inhibiting composition comprises one or more active agents selected from the group consisting of small molecule inhibitors, antibodies, antisense RNA and interference nucleic acid sequences.

Interference RNA in mammalian systems requires the presence of short interfering RNA (siRNA), which consists of 19-22 nt double-stranded RNA molecules, or shRNA, which consists of 19-29 nt palindromic sequences connected by loop sequences. Down regulation of gene expression is achieved in a sequence-specific manner by pairing between homologous siRNA and target RNA. A system for the stable expression of siRNA or shRNA was utilized to generate transgenic animals (Hasuwa et al. FEBS Lett 532, 227-30 (2002), Rubinson et al. Nat Genet 33, 401-6 (2003) and Carmell et al. Nat Struct Biol 10, 91-2 (2003)) and can be used in accordance with the present invention to produce animals whose fertility can be regulated. A conditional interference RNA-based transgenic system would provide the additional benefit of being able to control the level of gene expression at any given stage during the life of the animal.

Recently, a biological theory has been advanced which postulates that the combinatorial nature of histone modifications provides a mitotically and meiotically heritable information storage mechanism, i.e. the “histone code”, which dictates correct spatial and temporal gene expression patterns in organisms by regulating access to underlying DNA. Post-translational modifications of core histone n-terminal tails including acetylation, phosphorylation, poly(ADP-ribosylation), ubiquitination, and methylation.

Histone modifications which accumulate on chromatin during gametic differentiation must be removed and replaced with embryonic histone modifications to allow for successful reprogramming. If, as the histone code suggest, each histone modification is contingent upon preceding modifications, then initial modifications in the early embryo will likely effect subsequent modifications in the embryo, fetus, and possibly into adulthood. Therefore endogenous and environmental factors effecting these initial histone modifications may have a tremendous impact on human health.

Animal cloning experiments have demonstrated that the maternal factors which are responsible for reprogramming somatic cell nuclei localize to the egg cytoplasm. Based on this observation, one might predict that the chromatin modifications most affected by the egg cytoplasm may prove to be most relevant to reprogramming events. To test this hypothesis, applicants investigated global changes in histone modifications in the egg and early embryo by indirect immunofluorescence and found that, as opposed to serine 1 H4 phosphorylation, lysine 4 H3 methylation, lysine 9 H3 methylation, and polylysine H4 acetylation, staining for methylated arginine 3 H4 and arginine 17 H3 was absent during metaphase stages of the egg and early embryo.

DNA methylation represents one type of chromatin modification known to undergoe genome-wide reprogramming in germ cells and early embryos. However, a demethylating enzyme has yet to be identified. Applicants have reason to believe that ePAD may be functioning as a demethylating enzyme. ePAD mainly localizes to an abundant cytokeratin intermediate filament structure unique to the cytoplasm of the mammalian egg and early embryo, however, ePAD is also localized to a lesser extent in the cell nucleus. PADs are calcium dependent sulfhydryl enzymes whose known in vivo substrates include keratin and, more recently, the core histone proteins H2A, H3 and H4.

Catalyzed by PRMT1, arginine 3 H4 methylation represents a rare histone modification that is associated with nuclear receptor activation and cellular differentiation. A comparison of the substrate specificity of PRMT1 with the epithelial cell PAD known to deiminate cytokeratin reveals that the activity of both enzymes is specifically directed toward the guanidino group of arginine residues which are flanked by multiple glycine residues. In particular, the substrate sequence of peptidylarginine deminase deimination in keratin has been confirmed by HPLC and is shown below (Deiminated arginine is underlined):

C-terminal tail of mouse keratin K1.

SGGSYGGSSGGGRGGSSSGGGGVK (SEQ ID NO:10). This finding has led applicants to propose that ePAD possesses a histone arginine demethylase activity in addition to a potential cytokeratin deiminase activity.

To confirm such activity, the ability of a commercially available skeletal muscle PAD to demethylate methylated arginine 3 on histone H4 was investigated by Western blot analysis. A complete loss of staining with antibodies to this modification was observed following PAD treatment. These and other preliminary results support the hypothesis that PADs in general may function as demethylases. More specifically, ePAD is believe to be a peptidylarginine deiminase with deiminating and potentially demethylating activity towards the gly-arg-gly motif found in keratin and the core histone proteins H4 and H2A. Thus ePAD may represent a prominent maternal epigenetic regulator of early development.

In accordance with one embodiment of the invention a method is provided for removing postranslational marks from core histone proteins in vivo. More particularly, in one embodiment the method results in the removal of methyl groups from arginine residues located on the n-terminus of the core histone proteins H4 and H2A or from lysine residues located on the n-terminus of H3 and H4. The method comprises the step of introducing PAD activity into a target cell. In one embodiment a recombinant nucleic acid sequence encoding a PAD protein, preferably the ePAD polypeptide of SEQ ID NO: 1 or 3 is introduced into the cell. The cells can be transiently transformed or the construct can be intergrated into the cells genome. The PAD protein can be expressed through the use of a constitutive promoter or an inducible promoter. Means for introducing nucleic acid sequences into cells are well known to those skilled in the art.

Alternatively, the PAD protein itself can be introduced into the cell. In addition, Ca++ can also be introduced into the cell, either by contacting the cell with an exogenous source of Ca++, direct injection of Ca++ or contacting the cells with an agent that stimulates cellular uptake of Ca++. Methods for introducing proteins into cells include but are not limited to microinjection, electroporation, calcium chloride premeablization, polyethylene glycol permeabilization, protoplast fusion or cationic lipid premeablization. In one embodiment a protein is introduced into the cell through the use of the Bioporter protein delivery system (GeneTherapy Systems, Inc. San Diego, Calif.). In accordance with one embodiment ePAD activity is introduced into a primary cell culture or a cell line to remove postranslational marks from the cell's genome and thus enhance the cell's ability to divide and enhance the cell's totipotency.

To further test the hypothesis that accumulated covalent histone tail modifications that encode gametic gene expression patterns must be specifically removed and replaced by histone modifications which will dictate embryonic gene expression patterns, the effect of egg cytoplasm on somatic cell histone modifications was investigated. More particularly, somatic cell nuclei were isolated from cells of differentiated tissues and were microinjected into mouse oocytes. The eggs were fixed at various time points, and then stained with the anti-modified histone antibody panel. DNA staining of the oocyte confirmed the presence of the microinjected nuclei. When a cumulus cell nucleus was microinjected into a mouse oocyte and allowed to incubate, staining with an antibody specific for SGR*GKGGKGC (SEQ ID NO: 6; the H4R3M2 antibody) revealed that the signal was lost in cells incubated longer than two hours. As a control, the staining pattern for an antibody specific for the epitope S*GRGKGGKGC (SEQ ID NO: 9), wherein the serine residue is phosphorylated (the PS-1 antibody), was found not to change after incubation of the cell for two hours. Thus the modification of the chromatin does not involve the removal of the terminal histone amino acids. Similarly when the peptides SGR*GKGGKGC (SEQ ID NO: 6) and S*GRGKGGKGC (SEQ ID NO: 9) were injected into the eggs the H4 methylated arginine modifications were removed within one hours time whereas the staining remained for the antibody (PS-1) directed against SEQ ID NO: 9.

In accordance with one aspect of the present invention it is believed that ePAD functions to remove posttranslational modifications from histones and is involved in restoring totipotency to the embryo. This activity can be used to rejuvenate cell lines to stimulate them to divide and behave more like true stem cells. The method comprises introducing ePAD activity into a cell and culturing the cell in the presence of Ca++.

Because the PAD enzymes are believed to play a role in modifying postranslational modification of histones, which in turn impacts transcription and DNA sysnthesis activities, it is anticipated that these enzymes may be involved in certain cancers. Accordingly, one embodiment of the invention is directed to the used of PAD and in particular ePAD as a diagnostic marker for neoplastic disease such as cancer. The method would comprise the steps of screening for elevated levels or inappropriate expression of PADs, including the expression of e-PAD in somatic tissues.

### EXAMPLE 1

ePAD is Present in Human Oocytes and Embryos Using Indirect Immunofluorescence.

The immunofluorescent localization of ePAD in human eggs and an 8 cell human embryo using antibodies to mouse recombinant ePAD was investigated.

Methods:

Mature, metaphase II eggs and 8-cell embryos were obtained from the Human Gamete and Embryo laboratory and fixed in 4% paraformaldehyde for 20 min at room temperature. Eggs were then washed and permeabilized in 0.5% Triton X-100 for 20 min at room temperature, washed and blocked in PBS containing 1% BSA and 5% goat serum for 30 min. Eggs and embryos were then washed and incubated with 20 μg/ml of either purified anti-ePAD IgG antibody or preimmune IgG for 2 h at room temperature. Eggs and embryos were then washed and incubated for 1 h in a 1:300 dilution of Texas Red labeled goat anti-guinea pig IgG.

Embryos and oocytes were then washed and placed in 0.4 mg/ml RNase in PBS with 1% BSA for 30 min and incubated in 20 nM Sytox chromatin stain (Molecular Probes, Eugene, Oreg.) for 10 min. Embryos and oocytes were then extensively washed, placed in slow fade (Molecular Probes, Eugene, Oreg.) equilibration media for approximately 1 min and then mounted on slides in slow fade mounting media.

Images were obtained on a Zeiss 410 Axiovert 100 Microsystems LSM confocal microscope. For each panel, attenuation, contrast, brightness and pinhole aperture remained constant. Four second scans were averaged four times per line using a 63× oil lens equipped with a zoom factor of two, and attenuation, contrast, brightness and pinhole aperature remained constant.

Results:

Cytoplasmic staining was seen in the metaphase II egg and the 8-cell embryo incubated with ePAD IgG. No staining was evident when eggs/embryos were incubated with preimmune IgG.

Conclusion:

ePAD is abundant in the cytoplasm of human oocytes and embryos as evidenced by indirect immunofluorescence.

### EXAMPLE 2

ePAD is Present in Paraformaldehyde-Fixed Human Ovarian Cross-Sections.

Methods:

Paraformaldehyde-fixed 10 micron cross-sections of normal human ovaries (InnoGenex, San Ramon, Calif. 94583) were processed for indirect immunofluorescence. Slides were immersed in a descending xylene/ethanol series then washed in phosphate buffered saline (PBS). Slides were blocked in 100 ul droplets of 3% bovine serum albumin (BSA)/PBS containing a 1:10 dilution of normal goat serum for 60 minutes at room temperature. A 1:50 dilution of the primary antisera (either guinea pig anti-ePAD sera or pre-immune sera) in 3% BSA/PBS was added to each ovary cross-section in 100 ul droplets then incubated overnight at 4° C. Slides were washed in PBS-Tween 20 followed by PBS. Subsequently, 100 ul of goat anti-guinea pig IgG-FITC (1:200 dilution in 3% BSA/PBS) was placed on each ovary cross-section and incubated for 90 minutes in the dark at room temperature. Slides were washed in PBS then mounted with Slo Fade mounting media (Molecular Probes, Eugene, Oreg.) and stored at 4° C. until visualization. Slides were viewed using a Zeiss inverted fluorescent phase contrast microscope.

Results:

Cytoplasmic staining of primary follicles was seen in the ovary cross-sections incubated with ePAD sera. No staining was visible when ovary cross-sections were incubated with the pre-immune sera.

Conclusion:

ePAD is present in primary follicles in human ovarian tissue as evident by indirect immunofluorescence.

1. An antibody that specifically binds to an ePAD polypeptide.

2. The antibody of claim 1 wherein the antibody specifically binds to the polypeptide of SEQ ID NO: 1.

3. The antibody of claim 1 wherein said antibody is a monoclonal antibody.

4. A composition comprising the antibody of claim 1 and a pharmaceutically acceptable carrier.

5. A method for identifying antagonists of ePAD activity, said method comprising
contacting an ePAD protein with a methylated peptide substrate and a potential ePAD antagonist, to form an assay composition;
incubating the assay composition, under conditions permissive for ePAD demethylase activity in the absence of an ePAD inhibitor; and
identifying antagonists of ePAD activity based on the ability of said potential ePAD antagonist to decrease the demethylase activity of ePAD.

6. The method of claim 5 wherein the antagonist activity is detected through the use of an antibody that specifically binds to either the demethylated peptide substrate or the methylated substrate.

7. The method of claim 5 wherein a control assay composition is prepared comprising the ePAD protein and methylated substrate but lacking the potential ePAD antagonist, and wherein said identifying step comprises determining the relative amounts of methylated substrate present in the assay composition compared to the control assay composition after said incubation step, wherein the existence of a higher amount of methylated substrate in the assay composition relative to the control assay composition identifies an antagonist of ePAD.

8. The method of claim 5 wherein the ePAD protein comprises an amino acid sequence of SEQ ID NO: 1.

9. The method of claim 8 wherein the methylated peptide substrate comprises an amino acid sequence of SEQ ID NO: 6.

10. A method of decreasing the fertility of a female mammalian species, said method comprising the steps of administering a composition comprising an inhibitor of ePAD activity.

11. The method of claim 10 wherein the administered composition comprises an antibody that specifically binds to ePAD.

12. The method of claim 10 wherin the administered composition comprises an amino acid sequence of SEQ ID NO: 1, or a fragment thereof.

13. The method of claim 10 wherein the administered composition comprises an antibody that specifically binds to the polypeptide of SEQ ID NO: 1.

14. A recombinant human ePAD gene construct, said construct comprising a non-native promoter operably linked to the nucleic acid sequence of SEQ ID NO: 2.

15. A transgenic cell comprising the construct of claim 14.

16. A method of screening for potential human therapeutic agents, said method comprising contacting a human ePAD polypeptide comprising the amino acid sequence of SEQ ID NO: 1 with a candidate compound under physiological conditions;
washing the human ePAD polypeptide to remove unbound and non-specific bound material; and
isolating compounds that remain bound to the human ePAD polypeptide.

17. The method of claim 16 wherein the human ePAD polypeptide is immobilized on a solid surface and the candidate is labeled.

18. The method of claim 17 further comprising the step of determining if the candidate compound selectively binds to said ePAD polypeptide relative to other PAD polypeptides.

19. An antigenic composition comprising an amino acid sequence comprising SEQ ID NO: 1 or an antigenic fragment thereof and a pharmaceutically acceptable carrier.

20. The composition of claim 19 further comprising an adjuvant.

21. The composition of claim 20 wherein the antigenic fragment consists of SEQ ID NO: 5 or a fragment thereof.
